(19) United States
(12) Patent Application Publication
(10) Pub. No.: US 2016/0058484 A1
(43) Pub. Date: Mar. 3, 2016

(54) INTRAMEDULLARY SUPPORT WITH POROUS METAL SPLINES

(71) Applicant: Wright Medical Technology, Inc., Memphis, TN (US)

(72) Inventors: Mary J. McCombs-Stearnes, Lakeland, TN (US); Scott A. Armacost, Germantown, TN (US)

(21) Appl. No.: 14/403,730

(22) PCT Filed: Aug. 26, 2014
(86) PCT No.: PCT/US2014/052716

§ 371 Date: Nov. 25, 2014

(51) Int. Cl. A61B 17/72; A61B 17/16

(52) U.S. Cl. CPC A61B 17/7291; A61B 17/1682; A61B 17/7283

(57) ABSTRACT

An intramedullary support for arthrodesis of a human midfoot, especially to correct Charcot deformity, is configured as an elongated beam or shaft having porous metal on an outer surface for bone ingrowth. For the medial column, the intramedullary support is emplaced in a K-wire guided bore extending through the metatarsal, cuneiform, and navicular bones into the talus. The beam or shaft can be polygonal in cross section and the porous metal can included particulate or trabecular metal arranged in discrete areas or along splines, such as titanium with a porosity comparable to that of cancellous bone. Splines or encircling lengths of porous metal can be flush or protruding from the surface of the beam or shaft, longitudinal along a cylindrical the beam, or oblique or wrapped helically, or on a beam of polygonal cross section. Bone ingrowth and ossification supports the medial column in alignment along the beam.

### FIELD

This disclosure relates to the field of surgical procedures and implants for fusing plural bones, in particular for fusing plural separate bones across one or more joints in the human midfoot, to improve anatomical alignment.

### BACKGROUND

Charcot midfoot deformity is a condition associated with diabetic neuropathy and lack of sensation in the extremities. A person with limited sensation can suffer a sprain, fracture, dislocation or similar damage to a foot during regular activities and be unaware of the injury, or unaware of the extent of the injury. Continued activities on the injured foot cause additional damage. The damage is progressive. A characteristic condition includes partial dislocation, fracture and misalignment of the metatarsal, cuneiform and navicular bones that form the midfoot. The normal arched shape of the midfoot along the successions of bones from the calcaneus to the distal phalanges, known as the midfoot “columns,” can collapse and in some cases assume a rocker bottom or rounded plantar side of the foot.

One way to ameliorate Charcot deformity is arthrodesis or fusion of the bones of the midfoot columns. The distinct bones can be re-aligned in a surgical procedure that may include resecting as well as fixing the successive bones to one another so that the bones fuse or ossify across abutting faces of the bones that formerly met at joints. A main load bearing column that is advantageously fused is the medial column (to the great toe). Two or more midfoot columns can be caused to fuse, such as the first and third metatarsal columns.

The procedure may include attaching one or more bracing plates along the exteriors of adjacent bones along the midfoot columns in need of support. The bracing plates are attached to the respective bones using screws. An alternative technique includes installing a longitudinal intramedullary nail or bolt as a supporting structure within the midfoot column. A compression screw from the metatarsal to the talus advantageously applies compression to urge the midfoot bones into engagement. Immobilizing the bones in that position permits them to fuse.

The shape as well as the alignment of abutting bones of a midfoot column can be modified. Spaces can be incised to receive wedges or spacers, or spaces can be excised and the adjacent bones brought together, e.g., to reverse the rounding of the foot and thereby achieve a more plantigrade contour. Patient harvested bone or allografts or synthetic materials capable of bone ingrowth can be inserted to supplement the degraded bones and joints and fill in structural stress points. The bones are held stationary, and after healing become fused or ossified. The object is at least to align the structures of the foot in a more nearly anatomical state, although there is a consequent loss of natural flexibility or relative freedom of motion.

Intramedullary supports also are known for fusing bone segments across a break, typically in a relatively large bone, such as a tibia, femur, humerus or the like. An elongated intramedullary support is placed in a longitudinally drilled bore forming a lumen in the bone. The support bridges between the segments of the bone across the break. The support is comprises an elongated shaft of stainless steel, titanium alloy or the like, variously termed a shaft, bolt, nail, screw or bar, etc. The shaft is smooth to permit the bone segments freedom to slide along the shaft and to abut one another endwise. Transverse screws can be inserted into the shaft through the bone to fix the relative positions of the bone segments and the intramedullary support. The intramedullary support may be an alternative to a bracing plate affixed externally to the broken bone segments by transverse screws. Or a bracing plate and an intramedullary support can be used concurrently.

The bones of the midfoot are smaller than the long bones of the arm or leg, although the metatarsals as elongated to an extent. The more proximal bones of the midfoot columns are block shaped. However, compression screws and other intramedullary supports are known for supporting bones in the midfoot in arthrodesis procedures. International publication WO 2004/014243-William discloses use of an elongated intramedullary nail for fixing the alignment of the first metatarsal, medial cuneiform, navicular and talus bones.

In such surgical procedures, for example considering arthrodesis of the medial column, the medial phalange is dislocated downwardly at the distal first metatarsal. The bones along the medial column are aligned while being drilled through with a pilot hole, from the distal first metatarsal into the talus. Alignment of the column may include excising a wedge extending laterally on the plantar side and opening downwardly, whereby closing the wedge reverses some of the downward arch in the medial column.

A K-wire or guide is inserted into the drilled hole and the alignment of the bones can be checked fluoroscopically. The hole is enlarged in diameter along the medial column, back to the talus, using a cannulated reamer guided on the K-wire. The talus is a primary base of structural support for the foot, carrying the tibia and fibula. The reamed bore has an inside diameter that accommodates the intramedullary nail with minimal clearance (e.g., 0.5 mm diametric clearance). The intramedullary nail is inserted through the entire medial column and into the talus at the proximal end, i.e., through the lengths of the first metatarsal, medial cuneiform and navicular bones, and proceeding about half the span of the talus.

In some cases, the intramedullary support can comprise a compression screw having a thread along the distal or pointed end threaded into the talus, and also a “headless” but externally threaded proximal end. The shaft is smooth over a distance between the threaded ends. The thread at the proximal end has a shorter thread pitch (less longitudinal advance per unit of rotation) than the thread on the distal part of the shaft extending into the talus, and the fastener length is selected such that the bones of the medial column are compressed against one another like pulling beads together along a string.

In alternative arrangements, such as the William example mentioned above, the entire length of the shaft is unthreaded and smooth. After the shaft is inserted into the midfoot column, lateral fasteners (screws or pins) are inserted through the respective bones and through transverse holes provided at spaced locations along the inserted shaft. In the example described in William, three transverse fasteners are used to affix the first metatarsal to the intramedullary shaft or “nail,” two fasteners to affix the talus, and one to affix each of the medial cuneiform and navicular bones. For the cuneiform and navicular bones, the transverse holes are slots with additional longitudinal clearance, permitting some longitudinal and/or rotational displacement of the bones held along the smooth shaft.

### SUMMARY

An object of this disclosure is to provide an improved intramedullary supporting beam or shaft for correction of Charcot midfoot deformities and the like. In particular, an elongated intramedullary support is provided with external surface areas carrying a hard porous material adapted for bone ingrowth. These surface areas can be strategically placed and spaced longitudinally, for example residing at the ends of the beam or shaft and/or being spaced along or around the beam or shaft. Porous areas spaced along the beam or shaft between smooth areas can be selectively placed to reside in the dense cortical tissue of the bones that are supported along the beam or shaft as opposed to less dense cancellous tissue. The porous surface areas also can be arranged to have a mechanical effect or to cooperate with the cross sectional shape of the beam or shaft. For example, the porous material can form splines or runners that provide mechanical engagement as well as surfaces apt for bone ingrowth. The beam or shaft can have a polygonal cross section with the porous material carried in areas that are at the junctions or between the junctions of the polygonal faces. The porous material can comprise particles or shaped trabecular pieces that are sintered onto the outside of the beam or shaft structure.

In some embodiments, spaced porous areas, splines or runners advantageously comprise Wright Medical Co. BIOFOAM® material or a similar material that is particularly apt for bone ingrowth. The BIOFOAM material includes irregularly shaped titanium elements that are fused at their surfaces by sintering, to provide a structurally robust thickness of porous reticulated material that secures the beam or shaft in cancellous or cortical bone and immobilizes the beam or shaft and the bones that are to be fused. As the bone heals, the bone tissue grows into the porous material to form a composite that supports the midfoot column.

BIOFOAM material is known for use in wedges and spacers, for example in Cotton osteotomies of the midfoot and Evans osteotomies in the rear foot, in each case structured as spacers or wedges inserted between bones, or into incised or resected bones, and affixed using supporting plates that are external to the bone and are held in place by screws driven through the plates and into the bones adjacent to the location of the wedge or spacer. For arthrodesis to ameliorate Charcot deformity, the BIOFOAM material facilitates bone ingrowth and incorporation of the supporting structure into the structure of the bone. The configurations described herein enhance engagement between the support and the bone, reducing the need for additional structures such as external support plates, lateral screws, compression threads and the like.

The intramedullary beam or shaft according to this disclosure is elongated and may have a cross section that is smoothly cylindrical or otherwise shaped. Certain embodiments are splined and certain embodiments have polygonal cross sections with the longitudinal apices or cusps between facets of the beam or shaft providing elongated edges that limit rotational migration. The porous metal material can comprise sintered particles, and in different embodiments is sintered to fuse with the body of the beam or shaft at the outer surface, or is wholly or partly embedded in a groove on the surface for mechanical fixation. The porous material can reside on the surface as a surface covering or can be arranged to be flush with the surface, or can protrude from the surface at elongated embedded splines. Areas of the porous material can be continuous or discontinuous, regularly or irregularly spaced, and optionally placed to engage with particular bone tissue types. For example, the porous areas can be located at either end of the shaft and/or at intervals along the shaft. The splines can extend longitudinally, obliquely or with a helical twist, along or between facets of a polygonal beam/shaft cross section.

The splines engage the bone along the inside surfaces of the elongated bore provided through the adjacent bones of the medial column, and reduce or prevent migration (longitudinal or rotational relative displacement of the bones and the beam or shaft). The BIOFOAM material is apt for ingrowth and with healing engages with and supports the bones of the medial column, whether used with or without supplementary transverse screws or pins or external supporting plates.

### BRIEF DESCRIPTION OF THE DRAWINGS

These and other objects and aspects will be appreciated by the following discussion of preferred embodiments and examples, with reference to the accompanying drawings, and wherein:

FIG. 1 is an X-ray depiction of an exemplary Charcot foot deformity, characterized by misalignment of the bones along a collapsed medial column of the midfoot.

FIG. 2 is a schematic view showing repair of the foot by embedment of an intramedullary beam according to the present invention, to fuse the first metatarsal, medial cuneiform, navicular and talus bone in anatomically correct alignment.

FIGS. 3 through 7 are views of alternative embodiments of the intramedullary beam.

FIG. 8 is a superior view of the repair shown in FIG. 2, the intramedullary having longitudinal splines.

FIGS. 9-11 are schematic illustrations of steps in a procedure including installing the intramedullary beam as described, the calcaneus being omitted in these views.

FIGS. 12-14 are perspective illustrations of additional alternative configurations of the intramedullary beam or shaft.

### DETAILED DESCRIPTION OF EXEMPLARY EMBODIMENTS

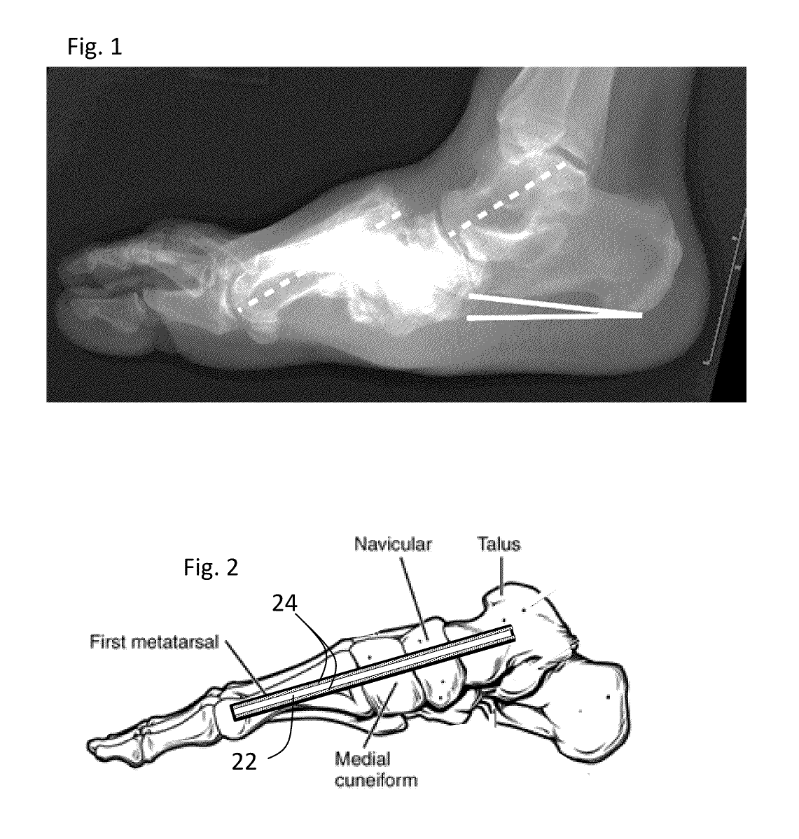

As seen in FIG. 1, in a Charcot foot deformity the normal alignment of the bones of the midfoot have been disrupted by dislocation and fracture. Apparatus and methods for repair of the deformity by arthrodesis can be applied to any or all of the midfoot columns, but are described, for example, with respect to the first metatarsal, the medial cuneiform and the navicular bone. These bones, together with the talus bone at the rear foot, are known as the medial column and normally provide much of the support needed for ambulation and other activities.

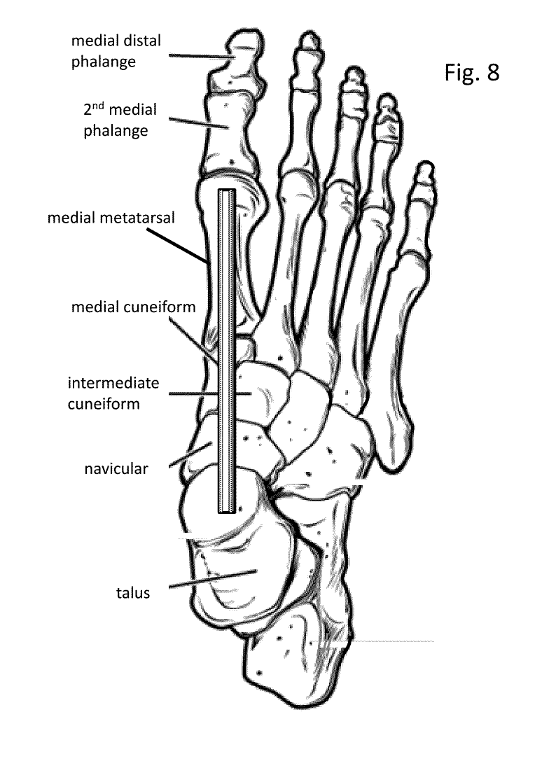

Charcot deformity can result from the accumulation of minor injuries that are not painful or perhaps are not noticed or fail to be regarded as serious, due to diabetic neuropathy and loss of sensation. An arthrodesis surgical procedure is indicated, for regaining reasonably anatomically correct alignment, which in FIG. 1 is to be achieved by bringing the bones along the two dashed lines into co-linear alignment. This is to be accomplished as seen in FIG. 2, by bringing the metatarsal, cuneiform and navicular bones into a line with the talus; forming a bore from the distal metatarsal into the talus; and inserting and embedding an elongated intramedullary beam or shaft 22, substantially complementary with the bore. FIG. 8 shows the result as in FIG. 2, but in a superior view.

It is an aspect of the invention that the external surface of the beam or shaft 22 is provided with at least one porous metal formation 24 that admits bone ingrowth for structurally affixing the beam to the respective bones. The beam fits closely against the internal surfaces of the bone. The porous metal formation advantageously comprises porous surgical metal material such as Wright Medical BIOFOAM. The porous material can have irregularly shaped titanium bodies affixed to one another and to the beam or shaft 22 as a substrate, to emulate the structure of cancellous bone for accepting bone ingrowth. The porous material is securely affixed to the substrate beam or shaft, for example by sintering of the particles to one another and to a one-piece integral substrate, or by integrally forming the substrate to include the porous areas at the surface, or by other affixation techniques. After ingrowth along the bones, the beam or shaft becomes structurally joined with the bone.

In a certain embodiments, the porous metal formation comprises BIOFOAM cancellous titanium, for example about 1.5 mm thick. This material is made from commercially pure Titanium and is readily fused to a Titanium or Titanium alloy shaft structure. BIOFOAM has a modulus similar to that of Tantalum (around 3 GPa) and a pore diameter of about 500 microns in a trabecular matrix architecture. BIOFOAM has a trabecular structure. Alternative embodiments can employ other forms of porous metal such as sintered beads or particles powders and other non-trabecular structures. Likewise, surfaces can be etched or otherwise treated to provide irregularities that support bone ingrowth.

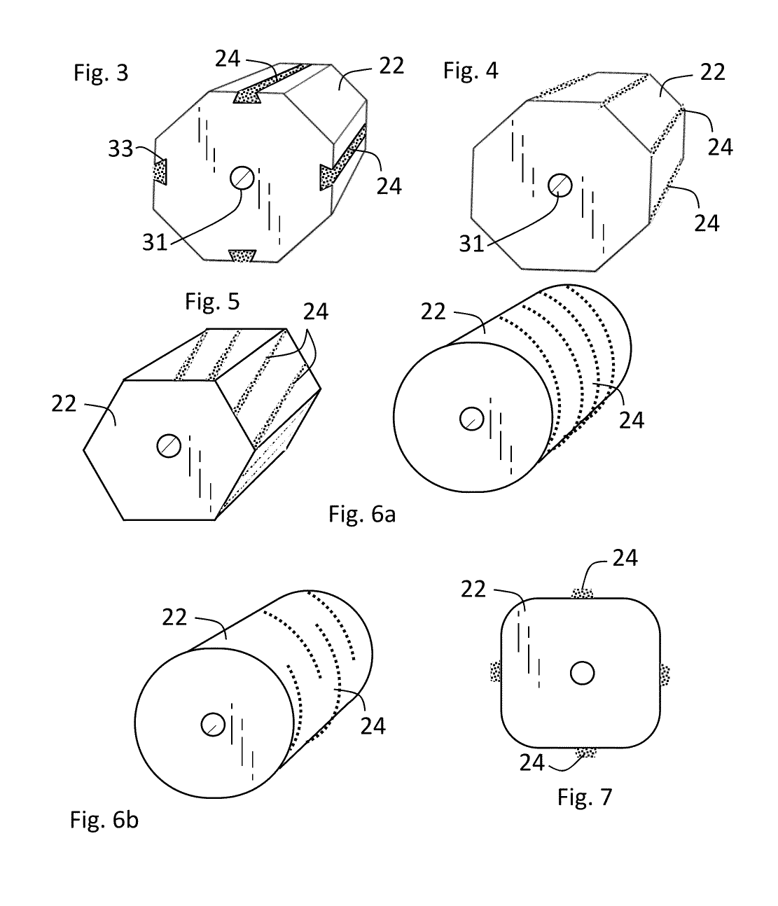

FIGS. 3 through 6 are perspective views showing exemplary alternative embodiments in which porous metal is arranged on the external surface of the intramedullary beam and thus reside against the inward facing surfaces of the bones when inserted in the bore. In a possible arrangement, the entire surface of the intramedullary beam can carry a coating of affixed particles to a predetermined depth, e.g., about 1 mm. Advantageously, however, the sintered metal particles can be applied at limited locations, especially as a plurality of splines on cylinders. The splines in the depicted embodiments extend wholly or partly along the length of the intramedullary beam. The cylinders are advantageously cannulated and in different embodiments can depart from a right cylindrical shape, for example so as to have a non-round cross section.

The main shaft portion of the intramedullary beam 22 can comprise a known surgical implant metal such as commercially pure titanium (CPTi) or cobalt chrome or a titanium alloy such as Ti₆AI₄V (titanium, aluminum and vanadium) or austenitic 316 stainless steel, etc. In the embodiments shown in FIGS. 3-5 and 7, the beam has a generally polygonal cross section and in FIG. 6, the beam is cylindrical. In these embodiments, the beam is cannulated at a central opening 31, which is useful for drilling, preparation of the bore and guiding the beam during insertion as discussed below.

Although the shaft of the beam comprises an elongated solid and the porous metal formation is provided on the external surface, there are several ways in which this can be accomplished. In FIG. 3 for example, the porous metal is mechanically affixed in axially parallel grooves 33, which are embedded into surfaces of the beam 22. Grooves 33 are trapezoidal in cross section but might be rectangular channels. In FIGS. 3 and 4, the cross section of the beam 22 is octagonal. In FIG. 5, a beam is shown with a hexagonal cross section and in FIG. 7, the cross section is rectangular. In these embodiments, porous metal formations are affixed on the outer surface of the beam or shaft 22. The porous formations can be wider or narrower in area, continuously elongated or discontinuous, regularly or irregularly sized and spaced. The positions of the porous areas can be chosen for example, to obtain selective attachment. In that event, porous areas can be located to correspond to the cortical tissue of bones to which the beam 22 is to be securely attached, while leaving smooth spaces between the porous areas to permit some longitudinal migration. It would be possible in a right cylindrical beam arrangement to permit rotational migration. However the depicted embodiments are arranged for rotational stability.

The grooves 33 are provided on every other face or facet of the octagonal cross section, thus providing four porous metal formation 24. Similar grooves 33 could be placed on all the eight facets or alternatively, fewer grooves could be used, for example at two diametrically opposite facets. FIG. 4 illustrates an alternative embodiment wherein the porous metal formations are axial splines corresponding to the cusps of the octagonal cross section (i.e., the longitudinal lines at which adjacent faces meet) instead of the facets. In FIGS. 4-6, the porous metal is applied to reside thinly the external surface. FIG. 7 shows that the porous material can form raised splines. In FIGS. 3, 4 and 7, the formations or splines 24 are parallel to the longitudinal axis. In FIG. 5 the formations 24 are inclined or oblique relative to the longitudinal axis, and in FIG. 6, the formations 24 are wound helically. In each case, the porosity of the formations 24 emulates cancellous bone. Ingrowth of bone tissue into the formations 24 when healing contributes to a secure structural connection and rotational stability where the respective bones are fixed permanently at a given position along the beam 24. Although temporary or permanent transverse screw or pins are possible (not shown), robust ingrowth of bone with the formations 24 achieves a similar effect.

As described, an intramedullary support for arthrodesis of a human midfoot at the medial column having metatarsal, cuneiform, navicular and talus bones, comprises an elongated beam 22 having a length substantially spanning a plurality of the bones of the midfoot, preferably from the distal metatarsal up to one third to two thirds and preferably half the span of the talus. The beam comprises a shaft with an external surface, and at least one porous metal formation 24 if provided on all or part of the external surface. The porous metal formation 24 admits bone ingrowth for structurally affixing the beam to the plurality of bones of the midfoot. Structurally similar midfoot beams can be placed in the other midfoot columns, such as in the medial and next lateral column or the first and third midfoot column.

The porous metal formation 24 advantageously comprises porous titanium configured to emulate cancellous bone, such as Wright Medical's BIOFOAM material. The porous metal can comprise one or more particular formations such a splines or longitudinal, oblique or helical areas on the surface of the shaft such as on the faces and/or cusps of a polygonal shape or annular collar areas, especially at the ends of the beam.

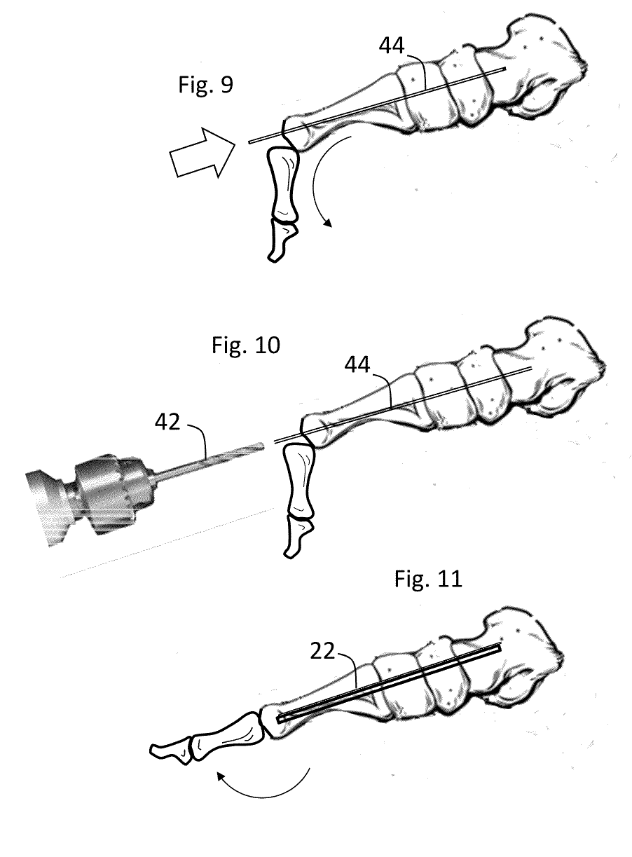

Exemplary steps in an associated method for surgical repair of a collapsed medial column of a human midfoot are shown in FIGS. 9-11. An initial step shown in FIG. 9, after gaining access through an incision (only the bones being shown) is to dislocate downwardly the second medial phalanx from the medial metatarsal, exposing the distal end of the medial metatarsal. A thin rigid rod 44 (known as a Kirschner wire or K-wire) is advanced from each bone to the next while holding the bones in position. The K-wire functions as a marker, a temporary holder and a guide. The K-wire enables a path to be formed and confirmed by fluoroscopic viewing, including locating the end of the path and measuring the length of the path and noting the placement of bones. A cannulated surgical drill 42 is applied over the K-wire to drill or ream a longitudinal bore along the K-wire that will receive the supporting column 22. Advantageously, the path is along the longitudinal center of the metatarsal and through the cuneiform, navicular and into the talus bones of the medial column.

Although not shown in FIGS. 9-11, for repairing Charcot midfoot deformity, it may be necessary or desirable to incise portions of the bones of the medial column and/or to insert wedges or spacers, so as to form a robust composite medial column structure wherein faces of the bones abut one another directly. Although not required in all cases, it may be desirable to include supplementary supporting structures such as an external fixator or plates (not shown) affixed to bridge across two or more of the bones of the medial column and across any wedges or spacers of bone, allograft or other material, which plates may be affixed with screws.

Drilling through the medial column (as aligned) proceeds into the talus, for example one third to two thirds of the thickness of the talus, to form a straight elongated bore generally coextensive with the longitudinal axis of the medial column and anchored in the talus. Advantageously, the K-wire guide rod 44 resides in place for guiding the cannulated surgical drill 42. The reamed bore is sized match the minor diameter of the intramedullary beam 22. The beam 22 is inserted as seen in FIG. 11, preferably being press-fit, thereby permanently fixing the medial column in alignment. The beam 22 is an elongated intramedullary support through the bore, spanning the medial column and terminating within the talus. The intramedullary support includes a at least one porous metal formation on an external surface of a shaft, as discussed above and shown in FIGS. 2 through 6. The dislocation of the phalange is repaired and the incision closed. After immobilizing the medial column for a period of healing, thereby subjecting the porous metal formation to bone ingrowth from the bones of the medial column, ingrowth of bone into the porous material 24, and ossification of the bones, forms the medial column to fuse into a unitary structure.

Referring back to FIGS. 3-6, the step of forming the intramedullary beam or shaft 22 includes placing the porous metal formation 24 on the surface of beam 22. Although the beam might be cylindrical and wholly coated with porous metal, it is advantageous to provide elongated splines and/or annular cylindrical (or polygonal) surface areas extending over a longitudinal length on the external surface of the beam 22, where porous metal formations 24 are presented to the surrounding bone tissue. The porous metal formation itself can be arranged in longitudinal strips that are flush with the surface of beam 22, nevertheless being exposed on the external surface of the beam for bone ingrowth. Alternatively the porous metal formation can provide splines that protrude radially from the surface of the shaft. The splines are preferably longitudinally continuous but also may be discontinuous with spaced gaps.

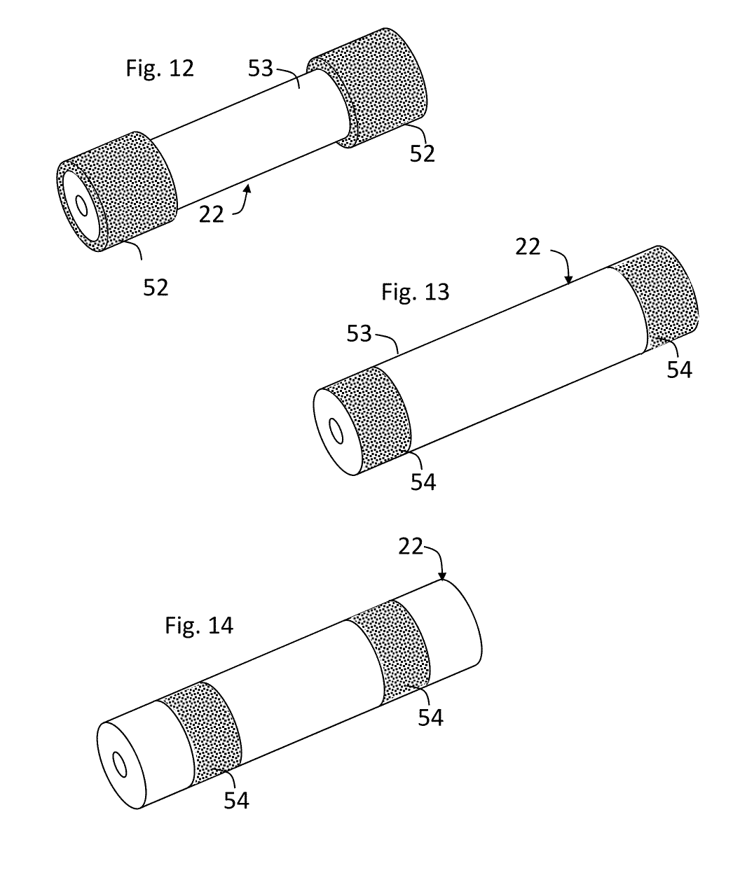

FIGS. 12-14 show alternative embodiments in which porous metal formations encircle the beam 22 and extend along a longitudinal distance. In FIG. 12, the ends of the beam 22 are provided with porous metal formations 52 that have a larger diameter than a smooth shaft portion 53. The larger diameter ends are force fitted into the talus and the distal metatarsal. The two portions 52 can be the same diameter and length or different diameters and lengths. Preferably the ends 52 are only slightly larger in diameter than shaft 53, the difference being exaggerated in the drawings. In FIG. 13, porous metal formations 54 are substantially the same diameter as the intermediate shaft 53.

In embodiments where the central length 53 is smooth and cylindrical, as in FIGS. 12-14, there is some freedom to migrate rotationally for bones at the central length. Apart from a cylindrical central length 53, a smooth surface provides some freedom for bones to migrate longitudinally. In the embodiments shown in FIGS. 3-7, however, the porous metal formations are configured as a plurality of splines atop or embedded in the external surface of the beam or shaft 22. The beam or shaft 22 has a polygonal cross section defining elongated faces that join at angularly spaced cusps or apices, and the splines 24 of porous metal can be longitudinal along the faces or along the cusps/apices. Or alternatively, the splines 24 can be oblique or inclined. These arrangements contribute rotational stability as well as the ability to hold the successive bone in alignment.

The porous metal arrangements as described can be employed on forms of beams or nails other than the simple lengths of metal shown in the drawings. For example, one or more porous formations as described can be provided on a compression screw, in particular over a part of a smooth shaft part of the compression screw between ends, either or both of which may be threaded.

The invention has been disclosed in connection with a number of alternatives intended to exemplify the subject matter. However the invention is not limited to the embodiments disclosed as examples. Reference should be made to the appended claims rather than the foregoing examples, in order to assess the scope of the invention in which exclusive rights are claimed.

What is claimed is:

1. An intramedullary support for arthrodesis of a human midfoot having bones defining a midfoot column, comprising:
an elongated beam having a length substantially spanning a plurality of said bones of the midfoot;
wherein the beam comprises a shaft with an external surface; and,
at least one porous metal formation on the external surface;
wherein the porous metal formation admits bone ingrowth for structurally affixing the beam to said plurality of bones of the midfoot.

2. The intramedullary support of claim 1, wherein the shaft of the beam comprises an elongated solid and the porous metal formation is affixed to an the external surface.

3. The intramedullary support of claim 1, wherein the porous metal formation comprises a trabecular material configured to emulate cancellous bone.

4. The intramedullary support of claim 3, wherein the porous metal formation comprises a plurality of splines on the external surface.

5. The intramedullary support of claim 4, wherein the splines extend parallel to a longitudinal axis of the shaft.

6. The intramedullary support of claim 4, wherein the splines are inclined relative to a longitudinal axis of the shaft.

7. The intramedullary support of claim 4, wherein the splines are wrapped around the shaft.

8. The intramedullary support of claim 3 wherein the shaft has a polygonal cross section defining faces meeting at angularly spaced cusps.

9. The intramedullary support of claim 8, wherein the splines are provided along the faces.

10. The intramedullary support of claim 8, wherein the splines are provided along the cusps.

11. The intramedullary support of claim 8, wherein the splines protrude from the external surface of the shaft.

12. The intramedullary support of claim 8, wherein the splines are embedded in the external surface of the shaft.

13. The intramedullary support of claim 1, wherein the beam is dimensioned and configured to encompass metatarsal, cuneiform, navicular and talus bones of a medial column in substantial anatomical alignment.

14. The intramedullary support of claim 1, wherein the beam is dimensioned and configured to encompass metatarsal, cuboid and calcaneus bones of a lateral column in substantial anatomical alignment.

15. A method for surgical repair of a collapsed column of a human midfoot, comprising the steps of:
aligning at least two bones of a midfoot column in substantial anatomical alignment;
drilling through at least part of the midfoot column as thereby aligned, to form a bore;
inserting an elongated intramedullary support through the bore to span the midfoot column,
wherein the intramedullary support includes a at least one porous metal formation on an external surface of a shaft;
immobilizing the midfoot column for a period of healing, thereby subjecting the porous metal formation to bone ingrowth from the bones of the midfoot column.

16. The method of claim 15, further comprising forming the porous metal formation to include an elongated spline on the external surface.

17. The method of claim 15, wherein the porous metal formation comprises a plurality of splines exposed on the external surface of the shaft.

18. The method of claim 15, wherein the porous metal formation comprises a plurality of splines embedded in the external surface of the shaft.

19. The method of claim 15, wherein the shaft has a polygonal cross section defining elongated faces that join at angularly spaced cusps.

20. The method of claim 15, wherein the intramedullary support is dimensioned and configured to encompass a midfoot column extending from a metatarsal to one of a talus in a medial column and a calcaneus in a lateral column.
